(19) United States
(12) Patent Application Publication
(10) Pub. No.: US 2024/0247217 A1
(43) Pub. Date: Jul. 25, 2024

(54) APPARATUS AND METHOD FOR GROWING BIOLOGICAL MATERIAL

(71) Applicant: BYRON BIOREACTOR TECHNOLOGIES PTY LTD, Byron Bay, New South Wales (AU)

(72) Inventor: Karl Alwyn LAWTON, Byron Bay (AU)

(73) Assignee: BYRON BIOREACTOR TECHNOLOGIES PTY LTD, Byron Bay, New South Wales (AU)

(21) Appl. No.: 18/576,681

(22) PCT Filed: Jul. 4, 2022
(86) PCT No.: PCT/AU22/50692

§ 371 Date: Jan. 4, 2024

(30) Foreign Application Priority Data
Jul. 5, 2021 (AU) 2021902035

(51) Int. Cl. C12M 1/00; C12M 1/26; C12M 1/34

(52) U.S. Cl. CPC C12M 29/06; C12M 29/04; C12M 29/22; C12M 31/10; C12M 33/08; C12M 41/12

(57) ABSTRACT

An apparatus for growing biological material, comprising a bioreactor chamber comprising at least one substrate positioned within an interior of the bioreactor chamber to support the growth of the biological material; at least one inlet to supply nutrient; and at least one outlet configured to enable biological material to be retrieved from the bioreactor chamber; a liquid nutrient container comprising at least one liquid nutrient outlet fluidly connected to the bioreactor chamber to supply a liquid nutrient; and an atomizer fluidly connected between the bioreactor chamber and the liquid nutrient container and comprising a carbon dioxide inlet fluidly connected to receive a source of carbon dioxide wherein the atomizer is configured to receive liquid nutrient from the liquid nutrient container and produce a liquid nutrient mist and provide a mixture of the liquid nutrient and the carbon dioxide from the carbon dioxide inlet to the bioreactor chamber.

### TECHNICAL FIELD

The present invention relates to an apparatus and method for growing biological material. In particular, an apparatus and method for growing algae.

### BACKGROUND ART

Algae, including cyanobacteria, is of high commercial value. It is among the most nutrient dense and easily digestible forms of nutrition, and algae species that are high in lipids, proteins, carbohydrates, peptides, anti-oxidants and other micro-nutrients are used in health supplements (for example spirulina, chlorella). Algae is also used as feed in aquaculture and animal husbandry, it can be used to grow pigments and medical agents and, if able to be grown efficiently, algae has potential in industrial as a biofuel and a source of bioplastics. Algae and in particular microalgae offer a wide range of valuable products. The whole biomass can be used for nutrition, aquaculture or as animal feed. Intracellular products like lipids, carbohydrates or pigments (chlorophyll, carotinoids, phycobilisomes) can be used for biodiesel or bioethanol production as well as dietary supplements, basic chemicals or as natural dyes.

Various value added products are produced by cyanobacteria, which have a consistently growing economic potential. In addition, they represent a rich source of polysaccharides (exopolysaccharides), vitamins, bioactive substances, lipids, fine chemicals (carotenoids, chlorophylls, phycobilisomes), amino acids, steroles, enzymes and pharmaceuticals. Cyanobacteria are also able to convert solar energy at high rates into selected energy sources such as lipids and bio-hydrogen. In order to cultivate these organisms efficiently, depending on the organism and the desired product, very different photobioreactors are sometimes used.

Actively ‘farmed’ algae cultivation methods traditionally include raceways and open ponds. The most widely used systems for microalgae cultivation in industrial scale are open ponds. Open ponds still are state of the art and for example the company Cellana (Kailua-Kona, Hawaii) founded in 2004 is successfully producing biomass and several products like e.g. polyunsaturated fatty acids (PUFAs) with such a system (http://cellana.com). Because such solutions are open-air systems, they lose a substantial amount of water to evaporation and do not efficiently use CO₂. In addition, raceways, in addition to being physically large, often suffer contamination from undesirable algae and microorganisms and can suffer low biomass concentration. Algal biomass grown from such systems is thus usually prohibitively uneconomic for most algae markets.

Growing Algae in water requires significant inputs of water and energy. Significant challenges are also created when attempting to grow algae on a large scale as light does not penetrate far into the water) Large systems therefore agitate the water to give algae access to light. Harvesting of algae grown in water is usually done by flocculation or other chemical extraction methods, filtering, centrifuging and other methods of extracting algae from algae-laden water which are energy intensive. Harvesting of suspended cells is still a problem of algal utilization. In E Molina Grima 1, E-H Belarbi, F G Acién Fernández, A Robles Medina, Yusuf Chisti. Recovery of microalgal biomass and metabolites: process options and economics Biotechnol Adv 2003 January; 20(7-8):491-515 it was reported that 20-30% of the total biomass production costs accrue during concentration of the diluted biomass as mentioned before.

Another current method of culturing algae is through the use of photobioreactors. These closed cycle solutions were designed to overcome the contamination and evaporation problems associated with open ponds. A typical photobioreactor has a number of transparent tubes usually less than 10 cm oriented to maximise sunlight capture. A microalgal water based ‘broth’ is circulated from a reservoir to these solar collecting tubes and back to the reservoir, with a portion of the algae being harvested after the solar collection tubes. This design relies on the reliable presence of sufficient levels of sunlight, and, because a photobioreactor is a closed system, oxygen produced during photosynthesis builds up until it inhibits algae growth and, therefore, the algal culture must be returned to a degassing zone where the excess oxygen is removed. In addition, photobioreactors require temperature maintenance and are very difficult and expensive to scale up. Consequently, photobioreactors are also usually prohibitively uneconomic for most algae markets.

More recently, attempts to reduce algae production costs have seen the introduction of nebulised designs such as that described in U.S. Pat. No. 8,722,389. Essentially this new type of bioreactor provides a nutrient ‘fog’, CO₂ and some form of light source to a ‘mat’ on which the algae will grow. Algae are generally 2-20 microns and absorbing nutrients carried in water requires significant effort on their behalf. Nutrients carried in a fog are more easily assimilated by algae and as a result the system requires 50-75% less nutrient solution than conventional water-based systems. While being much more volume and cost efficient than open systems or the photobioreactor, it comes attached to a number of its own problems. Most notably:
Surface tension effects in liquids mean that it is very difficult to nebulise them into the very fine droplets required in order to create a ‘fog’.
Liquid mists and fogs, once created, tend to further agglomerate and coalesce into bigger droplets which reduce the efficiency of uptake by the algae.
As these liquid agglomerations become bigger, the effects of gravity become increasingly problematic, requiring the use of a complicated series of sub-systems—firstly a reservoir to capture falling droplets, then a sub-system to re-nebulise the liquid, then another to return the new mist back to the algal mat.
While beneficial for the purposes of accelerating algae growth having high surface on the algal mat makes it simultaneously difficult to remove the algae. This in turn requires further complicated sub-systems to assist with the process of harvesting the algae.
The overall system consumes large quantities of energy, and consequently the economics of producing algae for most potential applications continues to be marginal.

The product concentration of industrial fermentation processes may achieve 100 g L-1 or more, but the biomass concentration of phototrophic processes does not reach such order of magnitude. Biomass concentration values range between 2 and 6 g L-1 in technical photo-bioreactors, whereas open-ponds are usually operated with low values of approximately 0.5 g L-1 because mixing as well as light and substrate supplementation are suboptimal in these systems.

There are also growth methods based on the use of biofilms. Biofilm-based systems generally require less pumping, and offer easier harvesting by scraping or vacuuming. However, current designs have largely focused on the raceway pond type of open-system, and as such don't address the evaporation and physical size problems associated with these system types. Using biofilms within phototrophic fermentation processes, one may overcome the bottlenecks which are associated with low cell concentration and in consequence low product concentration. Whereas reactor technology for utilization of phototrophic reactor technology forms biofilms can be described as mature for specific applications and may be transferred in a medium-term process to a larger scale, some technological setups are from an industrial perspective still in its infancy.

It will be clearly understood that, if a prior art publication is referred to herein, this reference does not constitute an admission that the publication forms part of the common general knowledge in the art in Australia or in any other country.

### OBJECT OF THE INVENTION

It is an object of the present invention to provide an improved apparatus and method for growing biological material that addresses or at least ameliorates the aforementioned disadvantages or provides a useful choice.

### SUMMARY OF INVENTION

The present invention is directed to an apparatus and method for growing biological material, which may at least partially overcome at least one of the above mentioned disadvantages or provide the consumer with a useful or commercial choice.

In particular, the present invention provides for an improved apparatus and method for commercial production of biological material such as algae with significantly lower energy consumption, significantly lower volume-per-mass of material-produced-per-unit-time, and a reduction in overall system complexity.

The invention is directed to the growing of biological material such as microalgae in a bioreactor 9

The apparatus is closed to prevent ingress of contamination such as non-compatible fungi and bacteria or algae predator species including copiapods and rotofers.

With the foregoing in view, the present invention in one form, resides broadly in an apparatus for growing biological material, comprising:
a bioreactor chamber comprising:
   at least one substrate positioned within an interior of the bioreactor chamber to support the growth of the biological material;
   at least one inlet to supply nutrient; and
   at least one outlet configured to enable biological material to be retrieved from the bioreactor chamber;
a liquid nutrient container comprising:
   least one liquid nutrient outlet fluidly connected to the bioreactor chamber to supply a liquid nutrient; and
an atomizer fluidly connected between the bioreactor chamber and the liquid nutrient container and comprising:
   a carbon dioxide inlet fluidly connected to receive a source of carbon dioxide
      wherein the atomizer is configured to receive liquid nutrient from the liquid nutrient container and produce a liquid nutrient mist and provide a mixture of the liquid nutrient and the carbon dioxide from the carbon dioxide inlet to the bioreactor chamber; and wherein the biological material is grown within the apparatus without exposure to water in its liquid form.

For the purposes of this specification, the term “biological material” includes unicellular microorganisms such as microalgae and cyanobacteria that utilize nutrients, water, air (in particular higher levels of carbon dioxide) and light to grow and multiply photo-autotrophically or utilize water, nutrients and air (in particular higher levels of oxygen) to grow heterotrophically in darkness or a mixture of these conditions to grow in mixotrophic conditions.

For the purposes of this specification, the term “mist” means a cloud of liquid droplets suspended in the air or a gas (such as carbon dioxide) and can encompass a fog or a vapor, or similar, where the liquid particles are between 2-50 microns in size.

In preferred embodiments, a plurality of substrates are positioned together vertically and horizontally within the bioreactor chamber to maximize production of biological material.

In preferred embodiments the substrate may comprise micro-scale geometric features such as honeycomb or fractal in order to maximise the surface area of the substrate via pores or channels for the adhesion and growth of the biological material. The substrate may be manufactured of a ceramic, polycarbonate, metal, plastic or natural material. The pores or channels in the substrate also facilitate light to penetrate the substrate and support growth of the biological material and can be varied in size depending upon the material (such as between 50-100 μm). The substrate is playing a pivotal protective role for the growth of unicellular microorganisms endowing the cells with a higher robustness towards critical physical and chemical conditions (e.g. salt condition, pH, disinfectants).

Preferably, the bioreactor chamber also comprises at least one relief valve configured to release excess oxygen produced by the biological material within the bioreactor chamber.

Preferably, the at least one outlet configured to enable biological material to be retrieved from the bioreactor chamber comprises a scraping means to enable biological material to be scraped from the bioreactor chamber.

Preferably, the liquid nutrient container also comprises at least one nutrient inlet configured to enable refilling of the liquid nutrient container with liquid nutrient.

The liquid nutrient itself is varied depending upon the type of biological material to be grown within the bioreactor chamber and can sourced from commercial suppliers. Nutrient Solutions are commercially available and are customised for each known species. The dosage of nutrient to the bioreactor maybe metered and automatically dosed.

In preferred embodiments a flow of liquid nutrient and carbon dioxide is maintained from the liquid nutrient container to the bioreactor chamber via an air compressor to create a Venturi effect to provide nutrient flow through the atomizer.

Preferably, the atomizer comprises a nozzle comprising an ultrasonic vibrator to induce the flow of carbon dioxide by the flow of the liquid nutrient within the atomizer. In preferred embodiments the ultrasonic vibrator produces a frequency range from 100 kHz to 120 kHz or 1500 kHz to 1900 kHz or 0.8 MHz to 3 MHz or 8 MHz to 9.8 MHz.

In preferred embodiments, the atomizer may take the form of a jet nebulizer, an ultrasonic wave transducer, humidifier or vaporizer.

Preferably, the apparatus for growing biological material also comprises a means for harvesting a portion of the biological material from the at least one substrate within the bioreactor chamber. More preferably, the means for harvesting a portion of the biological material may be a vibration assembly comprising a rod configured to connect to opposing sides of the bioreactor chamber and be connected at at least one end to a vibration means.

In preferred embodiments, the vibration means may be a frequency resonator which produces low amplitude sine waves to facilitate vibration of the rod and at least one substrate. However, other means for harvesting a portion of the biological material from the at least one substrate may be used such as sonification, acoustic vibration, piezoelectric vibration, water spray or chemical spray.

Preferably, the apparatus also comprises a light source configured to provide a uniform intensity of light to the at least one substrate. In this way the light source facilitates uniform photoautotrophic growth of biological material throughout the substrate.

The light source may comprise an high pressure sodium bulb, fluorescent light bulb, metal halide, LED fibre, OLED panel or natural sunlight. In preferred embodiments, the light intensity, photoperiod or light frequency inside the bioreactor chamber is varied dependent upon the species of unicellular organism to be grown in the bioreactor chamber.

Preferably, the apparatus for growing biological material also comprises at least one filter fluidly connected between the liquid nutrient container and the atomizer. In this way, particulate contaminants are removed from the liquid nutrient before passing to the bioreactor chamber in the form of a mist.

Preferably, the apparatus for growing biological material also comprises a delivery means configured to inoculate biological material on the at least one substrate within the bioreactor chamber. More preferably, the delivery means comprises a container for placing biological material to be delivered to the bioreactor chamber via a pump and nozzle assembly.

Preferably, the apparatus for growing biological material also comprises a temperature regulation means configured to provide heating and/or cooling to the bioreactor chamber. The temperature regulation means can take the form of an external heater or electrical conduction to the at least one substrate whereby the substrate is heated or cooled to facilitate optimal growth of biomass.

In preferred embodiments, a number of sensors positioned within the bioreactor chamber would be utilized to facilitate automated operation of the apparatus of the present invention such as temperature sensors and pH sensors. In addition timers and solenoids can be employed to provide automated harvesting. pH within the bioreactor chamber may be determined by a combination of the pH of the nutrient solution and carbon dioxide density

In use (as shown in FIG. 14), the apparatus for growing biological material is used to produce biological material by following the method steps of:
a. Sterilising the liquid nutrient container and the bioreactor chamber;
b. Coating the at least one substrate within the bioreactor chamber with a carbon source and water via the delivery means;
c. Seeding the at least one substrate within the bioreactor chamber with a seed biological material;
d. Providing a mist of liquid nutrient produced by the atomizer and carbon dioxide to facilitate growth of the biological material on the at least one substrate;
e. Harvesting a portion of the grown biological material from the at least one substrate;
f. Retrieving the released portion of the biological material from the bioreactor chamber via the at least one outlet; and
g. Repeating steps d. to f.

In preferred embodiments, the sterilization in method step a. is achieved via ozone passed through the liquid nutrient container and the bioreactor chamber.

In preferred embodiments, the sterilization in method step a. is achieved via ozone passed through the liquid nutrient container and the bioreactor chamber.

Preferably, the carbon source is carbon dioxide.

After harvesting a portion of the grown biological material in method steps e. and f. a seed amount of biological material is left on the at one substrate to facilitate regrowth in the next growth cycle at step d.

Harvesting can be performed at any time but will generally be determined by weight, thickness or other parameters which may be measured by timers, sensors or other devices.

The apparatus can be scaled as required from a small benchtop model for domestic or research purposes, to large scale commercial units. Multiple bioreactor chambers can be used in parallel to enable upscaling of production of biological material.

Any of the features described herein can be combined in any combination with any one or more of the other features described herein within the scope of the invention.

The advantages of the present invention include:
1. Improved efficiency of production of biomass product e.g. through large surface area of biomass growth versus size of apparatus or through improved assimilation of nutrient using nutrient mist and as a result requiring 50-75% less nutrient input than conventional water-based systems with a resulting lowered cost of production or through improved harvesting efficiency of biological material;
2. Improved scalability facilitating small or larger scale commercial production; and
3. Improved yield of product e.g. maintaining hygienic conditions within the closed apparatus increases productivity, reduces energy consumption and facilitates a continuous harvesting system (i.e. no need to clean out the whole system and start again).

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings by way of examples only as follows:

FIG. 1 shows a schematic plan view of a first embodiment of the present invention in the form of an apparatus for growing biological material.

FIG. 2 shows a schematic perspective view of the nutrient tank and bioreactor chamber of a second embodiment of the present invention in the form of an apparatus for growing biological material.

FIG. 3 shows a schematic perspective top view of a third embodiment of the bioreactor chamber of the present invention in the form of an apparatus for growing biological material.

FIG. 4 shows a schematic perspective bottom view of the third embodiment of the bioreactor chamber shown in FIG. 3.

FIG. 5 shows a schematic sectional perspective top view of a fourth embodiment of the bioreactor chamber of the present invention in the form of an apparatus for growing biological material.

FIG. 6 shows a schematic side view of a fifth embodiment of the bioreactor chamber of the present invention in the form of an apparatus for growing biological material.

FIG. 7 shows a schematic side view of a sixth embodiment of the bioreactor chamber of the present invention in the form of an apparatus for growing biological material.

FIG. 8 shows a schematic side view of a seventh embodiment of the bioreactor chamber of the present invention in the form of an apparatus for growing biological material.

FIG. 9 shows a schematic perspective view of the light source of an eighth embodiment of the present invention in the form of an apparatus for growing biological material.

FIG. 10 shows a schematic sectional side view of the bioreactor chamber of a ninth embodiment of the present invention in the form of an apparatus for growing biological material.

FIG. 11 shows a close-up schematic view of the bioreactor chamber of the ninth embodiment shown in FIG. 10.

FIG. 12 shows a schematic view of the light source of a tenth embodiment of the present invention in the form of an apparatus for growing biological material.

FIG. 13 shows a schematic view of multiple light sources of the tenth embodiment shown in FIG. 12.

FIG. 14 shows a process flow chart illustrating a method of use of the apparatus for growing biological material.

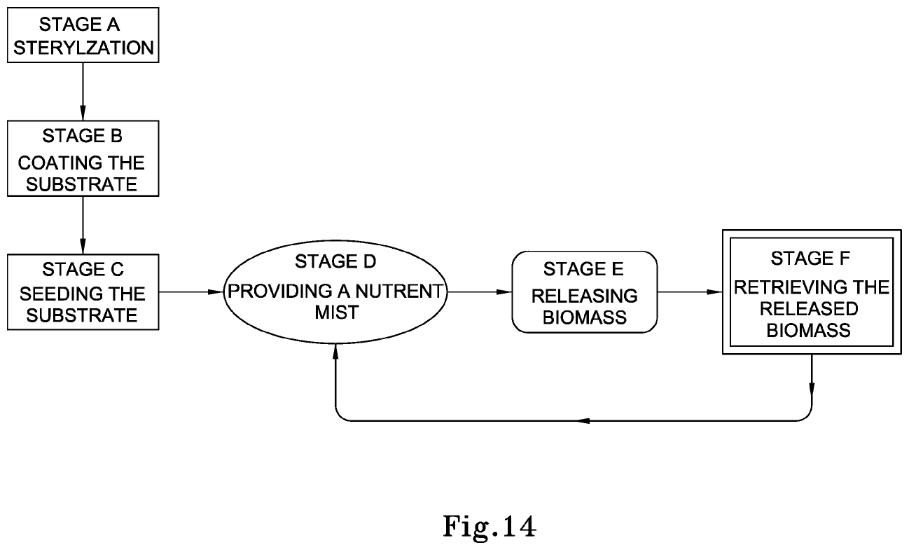

### DESCRIPTION OF EMBODIMENTS

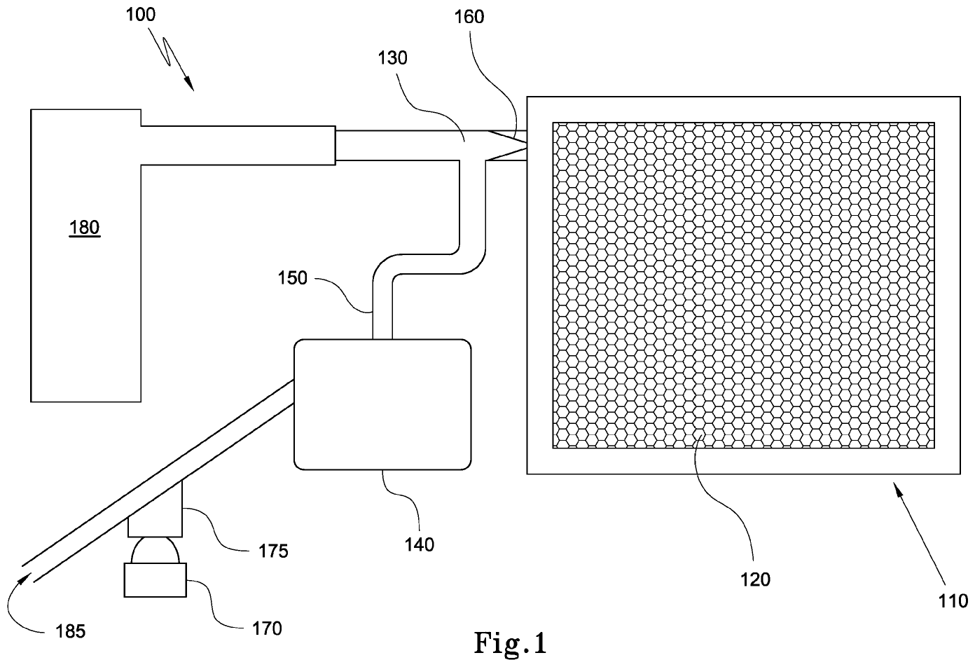

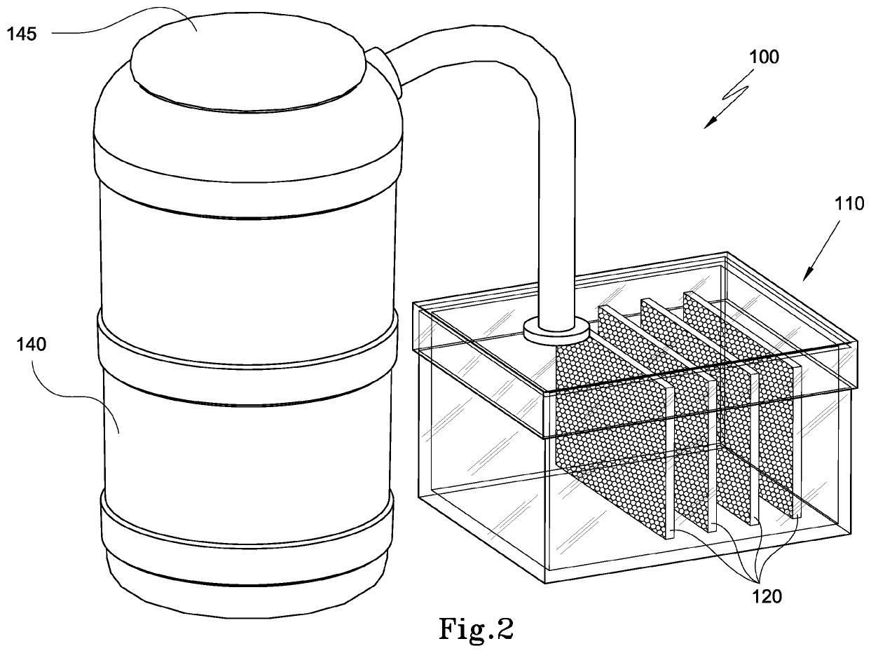

Referring to FIGS. 1 and 2, an apparatus for growing biological material of the present invention is generally indicated by arrow 100. The apparatus 100 comprises a bioreactor chamber 110 with at least one substrate 120, in the form of a plurality of polycarbonate honeycomb plates (best seen in FIG. 2) in order to maximise the surface area of the substrate for adhesion and growth of the biological material in the form of microalgae. The substrates 120 are positioned within an interior of the bioreactor chamber 110 best seen in FIG. 2. The bioreactor chamber 110 comprises an inlet 130 to supply nutrient: and an outlet in the form of a drain valve 190 configured to enable grown microalgae biomass to be retrieved from the bioreactor chamber 110. The Apparatus 100 can include a scraping means (not shown) positioned within the bioreactor chamber 110 in the form of a rubber edge configured with magnets to enable the scraping means to operate from outside the bioreactor chamber 110 to scrap grown biological material which adhered to the inside hard surface of the bioreactor chamber 110 to facilitate its retrieval from the drain valve 190 in conjunction with the harvesting means and a suction means configured to open when the atomizer 160 is closed in order to maintain positive pressure within the bioreactor chamber. The apparatus 100 also comprises a liquid nutrient container 140 with a liquid nutrient outlet 150 fluidly connected to the bioreactor chamber 110 to supply a liquid nutrient. The apparatus 100 also comprises an atomizer 160 in the form of a misting nozzle fluidly connected between the bioreactor chamber 110 and the liquid nutrient container 140 and comprising a carbon dioxide inlet 165 fluidly connected to receive a source of carbon dioxide 170 and a pump 175. The atomizer 160 is configured to receive liquid nutrient from the liquid nutrient container 140 to produce a liquid nutrient carbon dioxide mist which is delivered to the bioreactor chamber 110. The flow of carbon dioxide within the atomizer 160 is induced by the flow of the liquid nutrient. The flow of liquid nutrient and carbon dioxide is maintained from the liquid nutrient container 140 to the bioreactor chamber 110 via an air compressor 180 to ensure only positive pressure within the bioreactor chamber 110. The atomizer 160 creates a Venturi effect to draw the nutrient solution through from the liquid nutrient container 140 via the compressor 180. An atomizer nozzle can create fog or mist particle sizes from 6.5 um to 36 um depending on the pressure of the compressed air. The nutrient container 140 is also connected to a water supply 185. The bioreactor chamber also comprises at least one relief valve (not shown) configured to release excess oxygen produced by the biological material within the bioreactor chamber 110.

Referring to FIG. 2, the liquid nutrient container 140 also comprises at least one nutrient inlet in the form of lid 145 configured to enable refilling of the liquid nutrient container with liquid nutrient.

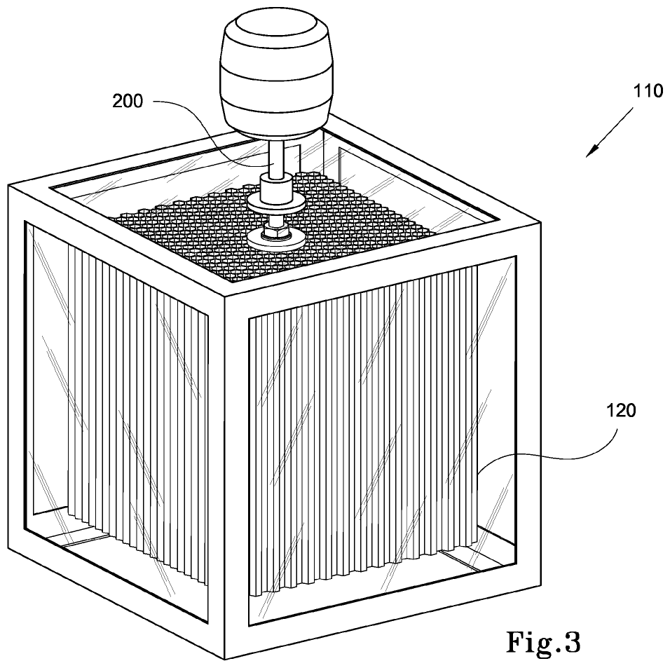

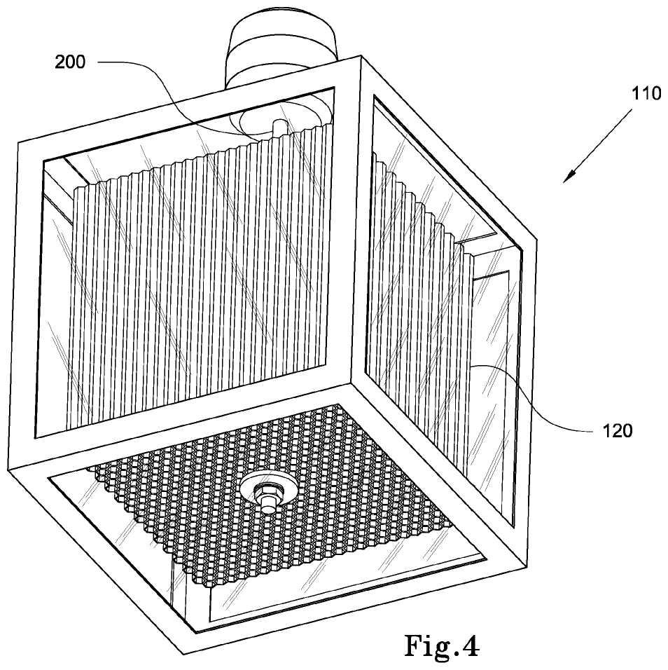

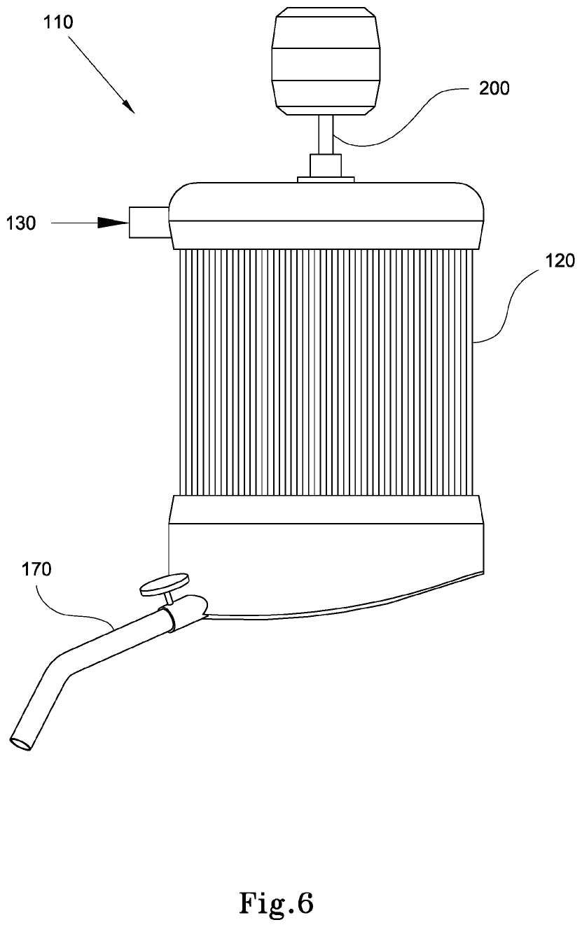

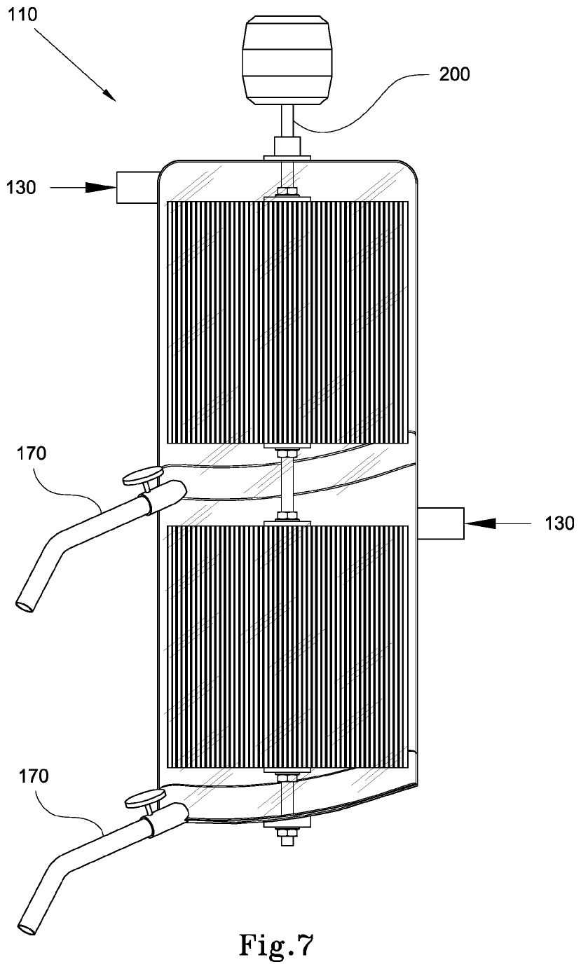

The apparatus 100 also comprises a harvesting means for harvesting the grown biological material from the bioreactor chamber 110 in the form of a centrally placed rod 200 which is made from titanium (shown in FIGS. 3 to 7) configured to pass through the substrate 120 and opposing sides of the bioreactor chamber 110 and be connected at one end to a vibration means such as a frequency resonator (not shown) at at least one rod end. The rod 200 can connect multiple stacked bioreactors 110 contained within the same bioreactor housing as shown in FIG. 7 which enables the stacked bioreactors 110 to be harvested as one unit to improve efficiency.

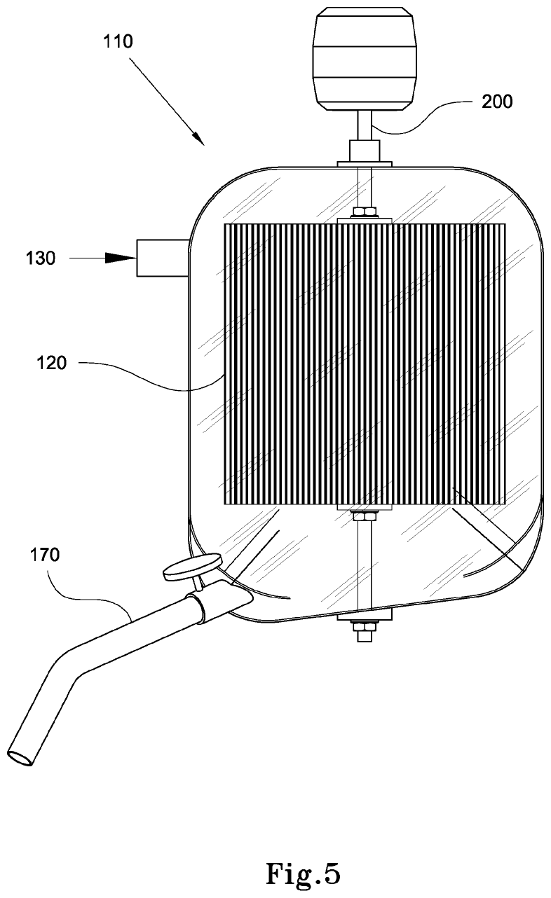

Referring to FIGS. 5-7, each bioreactor chamber 110 has an outlet in the form of a drain valve 190 in the bioreactor chamber 110 is configured to enable biological material to be retrieved from the bioreactor chamber and wastewater to exit the bioreactor chamber 110. The drain valve 190 comprises a suction means configured to open when the atomizer is closed in order to maintain positive pressure within the bioreactor chamber. Referring to FIG. 7, each stacked bioreactor chamber 110 can have each drain valve 190 connected to a common collection pipe and storage contained (not shown).

The housing of the bioreactor chamber 110 is made from polymer or metalized film (such as FEP (fluorinated ethylene propylene)) which is kept at a specific pressure via the at least one relief valve. During manufacture the housing is sealed by a sealing processes such as heat sealing or bonding, adhesives, metallising or thermoforming. The housing of the bioreactor chamber 110 has a non-stick coating on its inside surface to enable harvested microalgae to move to its lowest point where a drain valve 190 is situated for harvest of microalgae from the bioreactor chamber 110.

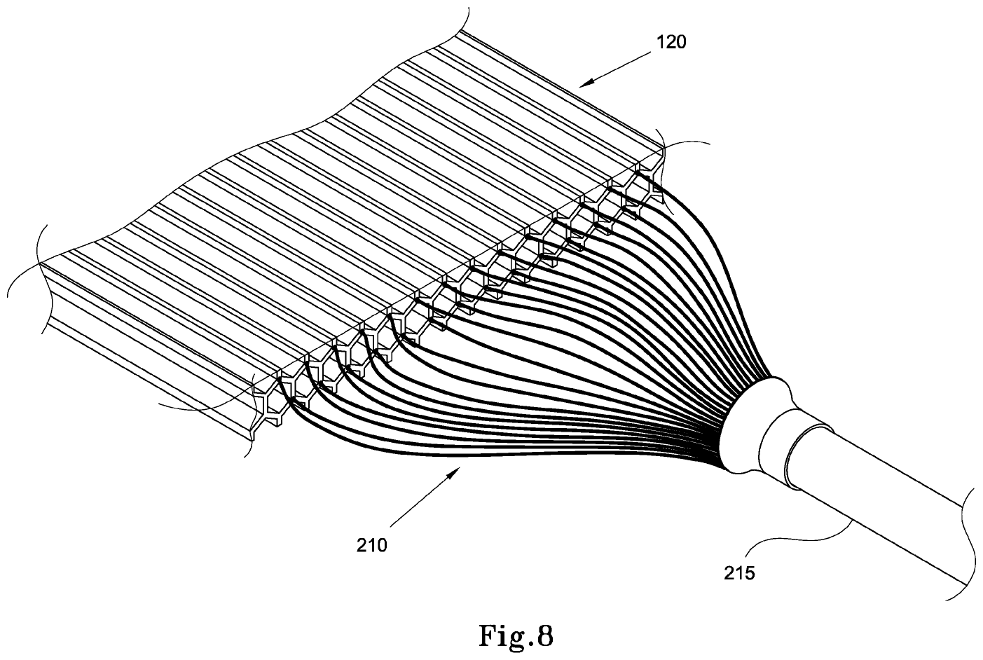

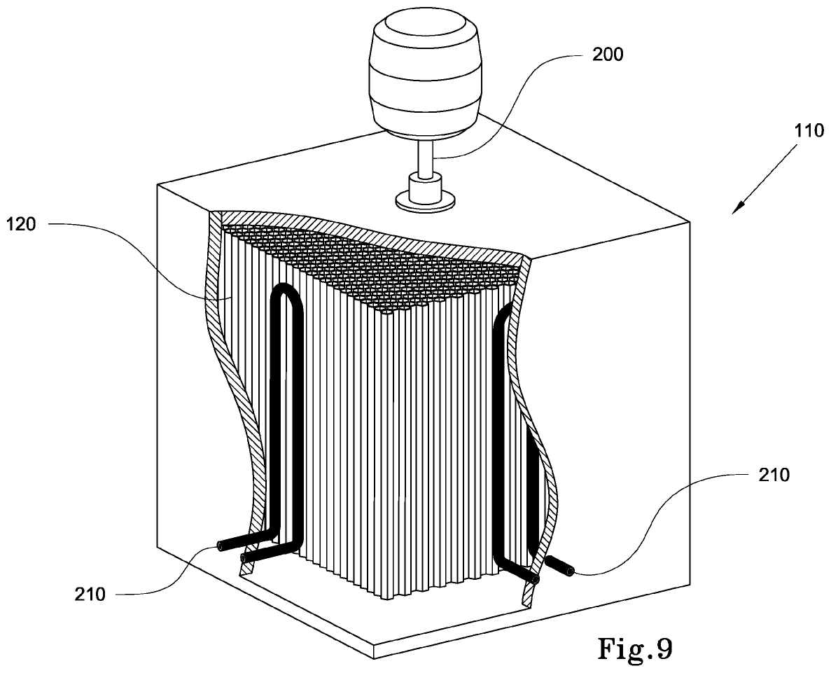

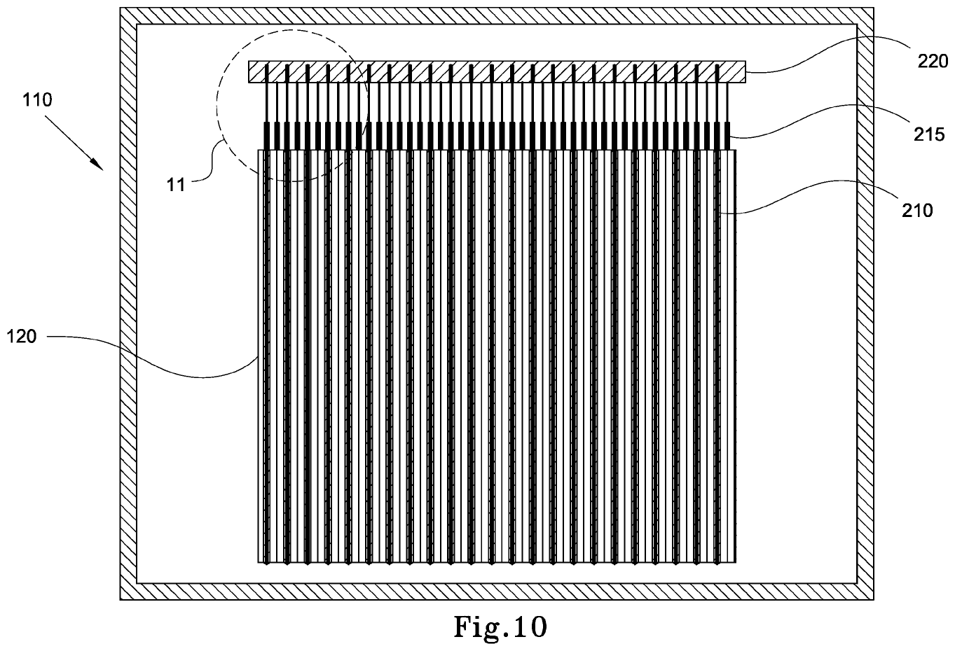

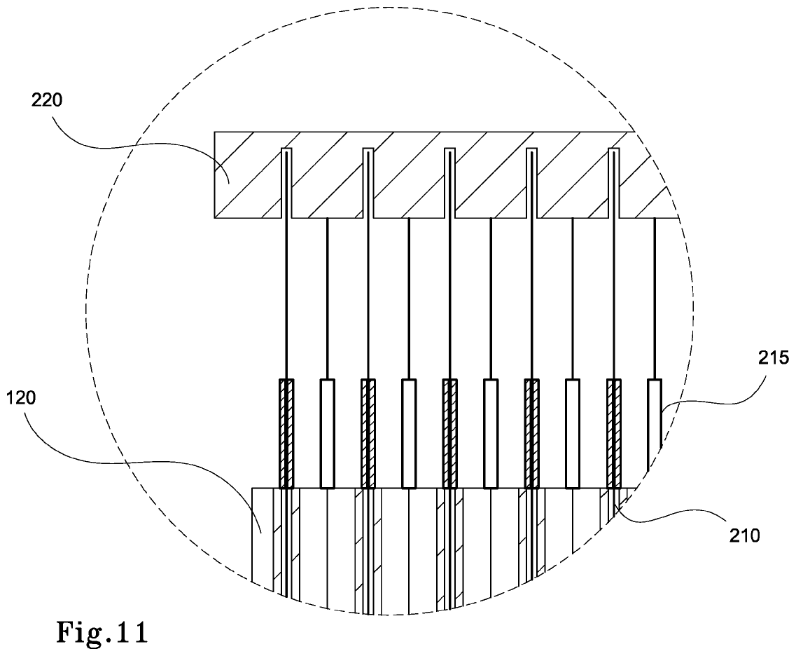

The apparatus 100 also comprises a light source in the form of at least one length of LED fibre 210 (as shown in FIGS. 8-11) configured to provide a uniform intensity of light to the at least one substrate 120 via integration into the substrate 120 to provide even light intensity throughout the substrate 120 to promote even growth of microalgae. The lengths of LED fibre 210 which exit the substrate 120 are bundled with heat shrink tubing 215 to stiffen and water seal the LED fibres 210. As shown in FIGS. 10 and 11, the lengths of LED fibre 210 exit the substrate 120 in the form silicone wire 215 and connect at positive and negative connectors to an electrical connection board 220 which can be situated within or outside the bioreactor chamber 110.

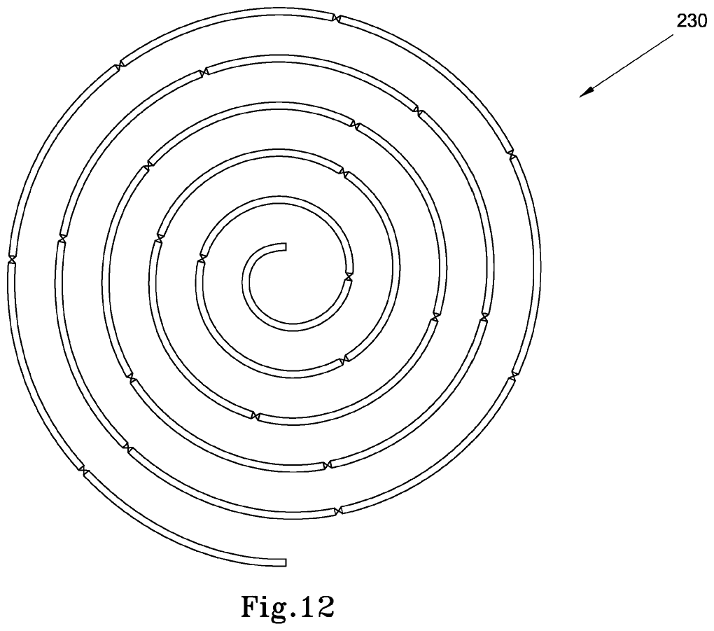

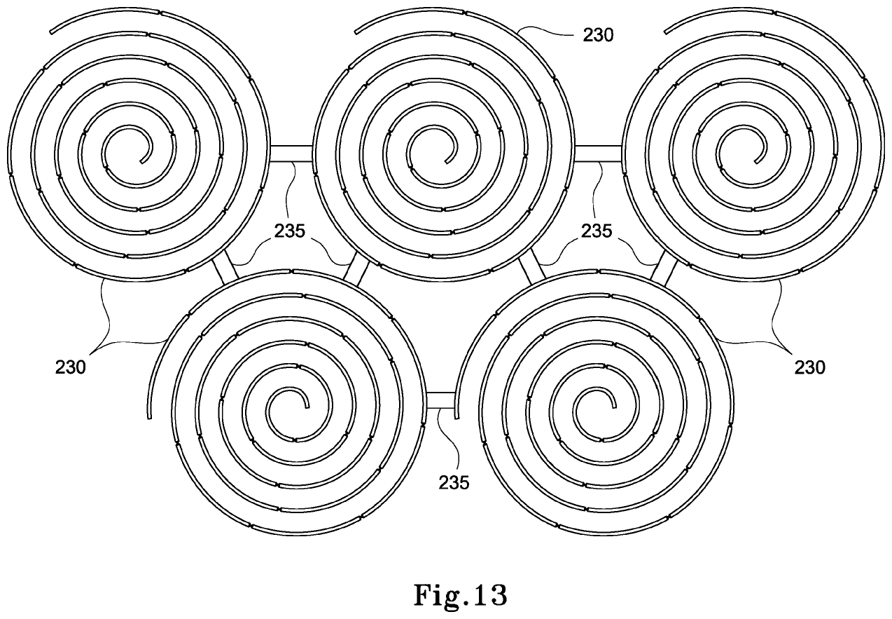

Alternatively, the light source is at least one flexible OLED panel 230 which is coiled into concentric circle form and which also functions as the substrate on which microalgae directly grows (as shown in FIGS. 12 and 13). As shown in FIG. 13, multiple OLED coiled panel 230 can be connected to one another at lock/removal points 235 to increase the surface area for growth of the microalgae. In this modular arrangement, the individual OLED coiled panel 230 can be replaced individually to save costs in maintenance of the apparatus 100. Harvesting of the microalgae is via the harvesting means rod 200 and frequency resonator.

The apparatus for growing biological material 100 also comprises at least one filter (not shown) fluidly connected between the liquid nutrient container 140 and the atomizer 160 to remove impurities from the liquid nutrient before it enters the bioreactor chamber 110.

The apparatus for growing biological material 100 also comprises a delivery means (not shown) configured to inoculate biological material on the at least one substrate within the bioreactor chamber. The delivery means comprises a container for placing biological material to be delivered to the bioreactor chamber via an air compressor and nozzle assembly.

The apparatus for growing biological material 100 also comprises a temperature regulation means in the form of a heater configured to provide heating and/or cooling to the bioreactor chamber 110.

Reference throughout this specification to ‘a preferred embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments. Similarly, the word “apparatus” is used in a broad sense and is intended to cover the constituent parts provided as an integral whole as well as an instantiation where one or more of the constituent parts are provided separate to one another.

1. An apparatus for growing biological material, comprising:
a bioreactor chamber comprising:
at least one substrate positioned within an interior of the bioreactor chamber to support the growth of the biological material;
at least one inlet to supply nutrient, and
at least one outlet configured to enable biological material to be retrieved from the bioreactor chamber;
a liquid nutrient container comprising:
at least one liquid nutrient outlet fluidly connected to the bioreactor chamber to supply a liquid nutrient; and
an atomizer fluidly connected between the bioreactor chamber and the liquid nutrient container and comprising:
a carbon dioxide inlet fluidly connected to receive a source of carbon dioxide
wherein the atomizer is configured to receive liquid nutrient from the liquid nutrient container and produce a liquid nutrient mist and provide a mixture of the liquid nutrient and the carbon dioxide from the carbon dioxide inlet to the bioreactor chamber; and wherein the biological material is grown within the apparatus without exposure to water in its liquid form.

2. The apparatus for growing biological material of claim 1, wherein the substrate comprises honeycomb in order to maximize the surface area of the substrate for adhesion and growth of the biological material.

3. The apparatus for growing biological material of claim 1, wherein the bioreactor chamber also comprises at least one relief valve configured to release excess oxygen produced by the biological material within the bioreactor chamber.

4. The apparatus for growing biological material of claim 1, wherein the liquid nutrient container also comprises at least one nutrient inlet configured to enable refilling of the liquid nutrient container with liquid nutrient.

5. The apparatus for growing biological material of claim 1, wherein the at least one outlet is a drain valve configured to enable biological material to be retrieved from the bioreactor chamber.

6. The apparatus for growing biological material of claim 1, wherein the atomizer comprises a nozzle and an ultrasonic vibrator configured to induce the flow of carbon dioxide by the flow of the liquid nutrient within the atomizer.

7. The apparatus for growing biological material of claim 1, wherein the apparatus also comprises a harvesting means for harvesting a portion of the biological material from the at least one substrate within the bioreactor chamber.

8. The apparatus for growing biological material of claim 7, wherein the harvesting means is a vibration assembly comprising a rod configured to connect to opposing sides of the bioreactor chamber and be connected at at least one end to a vibration means.

9. The apparatus for growing biological material of claim 1, wherein the apparatus also comprises a light source configured to provide a uniform intensity of light to the at least one substrate.

10. The apparatus for growing biological material of claim 9, wherein the light source is configured to pass through the at least one substrate and is selected from the group consisting of OLED light source, fiber optic filament and LED light source.

11. The apparatus for growing biological material of claim 1, wherein a flow of liquid nutrient and carbon dioxide is maintained from the liquid nutrient container to the bioreactor chamber via an air compressor to create a Venturi effect to provide nutrient flow through the atomizer.

12. The apparatus for growing biological material of claim 1, wherein the apparatus for growing biological material also comprises at least one filter fluidly connected between the liquid nutrient container and the atomizer.

13. The apparatus for growing biological material of claim 1, wherein the apparatus for growing biological material also comprises a delivery means configured to inoculate biological material on the at least one substrate within the bioreactor chamber.

14. The apparatus for growing biological material of claim 13, wherein the delivery means comprises a container for placing biological material to be delivered to the bioreactor chamber via an air compressor and nozzle assembly.

15. The apparatus for growing biological material of claim 1, wherein the apparatus for growing biological material also comprises a temperature regulation means configured to provide heating and/or cooling to the bioreactor chamber.

16. A method of using the apparatus for growing biological material of claim 1 comprising:
a. sterilizing the liquid nutrient container and the bioreactor chamber;
b. coating the at least one substrate within the bioreactor chamber with a carbon source and water via the delivery means;
c. seeding the at least one substrate within the bioreactor chamber with a seed biological material;
d. providing a mist of liquid nutrient produced by the atomizer and carbon dioxide to facilitate growth of the biological material on the at least one substrate;
e. releasing a portion of the grown biological material from the at least one substrate;
f. retrieving the released portion of the biological material from the bioreactor chamber via the at least one outlet; and
g. repeating steps d. to f.

17. The method of claim 16, wherein the carbon source is carbon dioxide.
